(19) United States
(12) Patent Application Publication
(10) Pub. No.: US 2011/0257482 A1
(43) Pub. Date: Oct. 20, 2011

(54) Laser Beam Collimation Apparatus

(76) Inventor: James K. Brannon, Leawood, KS (US)

(21) Appl. No.: 13/089,306

(22) Filed: Apr. 18, 2011

Related U.S. Application Data
(60) Provisional application No. 61/325,102, filed on Apr. 16, 2010.

(51) Int. Cl. A61B 1/00

(52) U.S. Cl. 600/117

(57) ABSTRACT

The present invention provides an endoscopic laser instrument for positioning the endoscopic instrument in relation to a reference point to measure the proper angular position of an associated medical device associated with a surgical site.

### CROSS-REFERENCE TO RELATED APPLICATION

This application claims the benefit of the prior filed U.S. provisional application No. 61/325,102 filed Apr. 16, 2010 which is incorporated herein by reference.

### FIELD OF THE INVENTION

The present invention is broadly directed to improvements in endoscopic surgery and, more particularly, to the use of a laser instrument to determine a field of surgical access such as within an endoscopic surgical site and to maintaining the collimation of a laser beam of such an instrument for effective use thereof.

### BACKGROUND OF THE INVENTION

Modern surgery tends toward minimally invasive techniques whenever possible. Although often more complicated in some ways for the surgeon, minimally invasive techniques result in a lower degree of trauma to the patient and less scarring because of much smaller and fewer incisions, thereby promoting faster healing and reducing possibilities for infections. In general, minimally invasive surgeries involve making one or more small incisions at appropriate locations and inserting tubular devices through the incisions to the surgical site. The tubular devices may be referred to as endoscopes, arthroscopes, and the like and typically have optical fiber based optical viewing apparatus and light sources, surgical instruments, lumens for exchanging fluids with the surgical site, or combinations thereof extending therethrough. In some circumstances it is more appropriate to separate the viewing scope with light source from specifically surgical instruments, thus requiring two incisions and endoscopes. This technique is sometimes referred to as triangulation.

The term “triangulation” can refer any one of a number of techniques which are used particularly in endoscopic surgery to perform a diagnostic or surgical act or operation and to monitor that operation from different angles, typically for the precise placement of instruments used in the operation. The principal forms of triangulation in endoscopic surgery include visual triangulation, tactile triangulation, and surgical triangulation. Visual triangulation refers to visual observation of the operation by the surgeon and typically includes the use of a viewing/light source endoscope. The viewing scope may be entirely passive, employing optical lenses and fiber optics, or it may include an electronic image array communicating a video image to a video monitor and recorder. Visual triangulation may also include various forms of radiant imaging, such as fluoroscopes, computed tomography, magnetic resonance imaging, ultrasound imaging, or the like. Tactile triangulation refers to the surgeon's use of tactile sense to recognize the impinging of an instrument on tissues, organs, other surgical instruments, or the like. Surgical triangulation refers to what the surgeon can actually reach using a given instrument from a given incision and established path to the surgical site. It should be appreciated that while the various forms of triangulation usually overlap, they are not necessarily identical. For example, a surgeon can often view more using visual triangulation than he can actually reach by surgical triangulation.

Endoscopic instruments are configured in a number of different ways, depending on their intended purpose. There are rigid endoscopes and flexible endoscopes. Rigid endoscopic instruments are preferred in situations when precise placement of an instrument is required, as for a surgical procedure. Some endoscopes are simply tubes or portal instruments which provide access to a surgical site for instruments which are passed through the scopes or for the exchange of fluids to and from the surgical site. Viewing scopes, including light sources, may be used for viewing a surgical site for diagnostic purposes or to view surgical operations occurring through the same scope or a different scope. Surgical operations may include cutting, shaving, debriding, cauterizing, or the like as well as grasping tissues or parts of organs, such as with forceps.

A problem which sometimes occurs, especially in hip joint surgery, is that the field of view greatly exceeds the field of surgical access, that is, the range of motion available to the surgeon using a rigid instrument. In hip joint surgery, the field of access is limited by the relatively small clearance between the acetabulum and the femoral head which has been distracted or pulled somewhat out of the acetabulum. Distraction of the femoral head from the hip joint is necessary to provide the physician with access to the joint surfaces. Once the femoral head is separated from the hip joint, access to various surface aspects of the hip joint and femoral head requires controlled movement of the patient's leg through a range of motion and fixation of the leg in selected positions. However, there is a limit to the surgeon's access to parts of the hip joint site from a given incision. Typically, the field of view is circular and provided by a triangulated scope. In contrast, the surgical access is somewhat conical in shape and may be elliptically conical, depending on the freedom of movement of the endoscopic instrument and the tissues and structures with which contact is to be avoided. It is particularly important to avoid unnecessary contact with the femoral head to minimize injury to the cartilage lining, since cartilage tends to have very limited capability of healing.

Additionally, during total knee replacement surgery it is desirable to insure alignment of the replacement knee along the tibal for proper orientation. Using an exterior alignment tool with a depending alignment marker to indicate the current alignment in relation to a desired alignment. One commercially available alignment tool includes a device adapted for receipt of an alignment rod. However, these alignment rods have a static length and typically are not projected to a fixed reference point in relation to the patient. Having an alignment device which indicates the current position as a projection to the patient to measure the correct orientation is desirable.

U.S. Provisional Application, Serial No. 6______, entitled LASER MEASURED FIELD OF ACCESS IN ENDOSCOPIC SURGERY, filed ______, 2010, by the inventor of the present application, and incorporated herein by reference, discloses the use of a low power visible laser beam to determine the field of access of surgical instruments at an endoscopic surgical site. A laser beam is a coherent beam of monochromatic light. Laser beams, as generated, usually have a low degree of divergence or, conversely, a high degree of collimation; that is, the radius of a laser beam does not increase significantly along its direction of propagation. However, the maintenance of such collimation requires a consistent medium along the propagation direction of the beam. Collimation of the beam can be disturbed by impingement of the beam on non-flat surfaces of substances having indices of refraction different from that of the initial medium through which the beam is initially propagated.

An endoscopic surgical site is often irrigated by a liquid medium to inflate the site to separate tissues for better viewing and access and to carry away any particles of tissue, blood, or the like resulting from surgical operations. A preferred liquid for such irrigation is normal saline solution which is approximately 0.9 or 0.91 percent sodium chloride solution (9 grams of sodium chloride per liter of water). Normal saline solution is isotonic with respect to human tissues; that is, it does not draw water out of tissues or cause water from the solution to be absorbed by the tissues by osmotic action. The presence of the irrigant within the surgical site or within endoscopic instruments through which the beam is propagated can disrupt the collimation of a laser beam by presenting a change in medium from air, by the presence of bubbles, by presenting surfaces of indeterminate and varying shapes to the beam, and the like. Even with a static volume of irrigant, distortion of the beam can occur by impinging on a meniscus within the laser instrument or the endoscope lumen. A meniscus is the curved surface of a liquid at a line of contact with the surface of a solid material. The radius of curvature of a meniscus depends on a number of factors, principally the degree attraction of molecules of the liquid to each other relative to their attraction to the molecules of the container. Within a small tube, the surface of a liquid in contact with surface can approach spherical in shape. If the laser beam loses collimation prior to reaching the endoscopic surgical site, it is less precise and, thus, less useful in accurately measuring a field of surgical access at the site.

### SUMMARY OF THE INVENTION

The present invention provides improvements in endoscopic surgery by the use of a laser instrument to enable a surgeon to visually estimate the limits of a field of surgical access within a field of view of an endoscopic surgical site and apparatus to maintain the collimation of a laser beam of such a laser instrument.

An embodiment of the invention provides a laser sighting endoscopic instrument or endoscope incorporating a laser unit for attachment to an endoscopic instrument in alignment with an axis of a lumen within a cannula portion of the instrument. The endoscopic instrument may, for example, be a portal instrument including a proximal hub with an elongated cannula extending therefrom. A lumen is formed through the hub and cannula toward a distal tip of the cannula. The hub has a socket formed at a rear port thereof which is configured to removably receive a self-contained laser unit. In one embodiment, the socket is threaded and a plug end of the laser unit is provided with complementary threading to enable the laser unit to be threaded into the hub of the portal instrument. The hub and laser unit, when joined, cooperate to position a laser beam from the laser unit along the longitudinal axis of the cannula. Alternatively, other types of junctions between the laser unit and portal hub are foreseen.

In one embodiment of the invention, the laser unit is provided with a laser generating element or laser source such as a laser diode, a power source such as a battery, and a control switch. It is foreseen that the laser unit could alternatively be powered by an external power source with a cable extending into the laser unit housing. The control switch can be a momentary switch for momentary activation of the laser by the surgeon or a latching or toggle type of switch which activates on a first press and deactivates on the next press of the button. The laser source is of such a character that the laser beam emitted therefrom is in the visible spectrum and bright enough for observation by the surgeon but low powered to avoid any heating of or other effects on tissues within the surgical site. It is also foreseen that the laser sighting endoscope can be used in conjunction with or incorporate a higher powered surgical laser unit to perform laser surgical procedures at the endoscopic surgical site.

In an embodiment of the invention, the laser unit includes a threaded tubular barrel extending beyond the laser source for engagement with a threaded rear port of the endoscopic instrument. A cylindrical lens is positioned within a bore of the barrel and completely fills the bore from the laser source to a distal end of the barrel. An embodiment of the lens has flat end surface at a proximal and a distal end which are oriented perpendicular to the axis of the laser beam. The distal end surface of the lens is flush with the distal end of the barrel to prevent the formation of a meniscus by contact of the surface by a liquid medium within the endoscopic instrument. The orientation and flatness of the end surfaces prevents refraction of the beam by end surfaces of the lens.

In an alternative embodiment of the invention, collimation of the laser beam is maintained by enabling substantially the entire bore of the threaded barrel to fill with an irrigant to prevent the formation of a meniscus within the bore which might distort the beam. The barrel is provided with air purge passages to enable air within the bore to be pushed out of the barrel as the irrigant enters the barrel to thereby prevent the formation of a meniscus. By this means, a consistent medium of propagation of the laser beam is provided.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is top plan view of an embodiment of a laser sighting endoscopic instrument or endoscope according to the present invention.

FIG. 2 is side elevational view of the laser sighting endoscope, shown partially in cross section and showing a diagrammatic cross sectional view of a laser unit for the instrument.

FIG. 3 is a fragmentary side elevational view at a somewhat enlarged scale and shows an internally threaded rear port of a hub of the endoscope to threadedly receive a laser unit therein.

FIG. 4 is a top plan view of an embodiment of a laser unit for use in the laser sighting endoscope.

FIG. 5 is an enlarged fragmentary diagrammatic cross sectional view of an end of an endoscopic laser unit and illustrates decollimation of a laser beam from the unit resulting from refraction by a meniscus of a liquid within a threaded barrel of the unit.

FIG. 6 is a view similar to FIG. 5 and illustrates an embodiment of an endoscopic laser unit including a cylindrical lens positioned within the threaded barrel of the unit to maintain collimation of the laser beam.

FIG. 7 is a view similar to FIG. 5 and illustrates an alternative embodiment of an endoscopic laser unit including a threaded barrel with air purge passages formed through the barrel to enable filling of a bore of the barrel with an irrigant.

FIG. 8 is a side elevation of an alternative embodiment of the endoscope with an internally received adapted.

FIG. 9 is a side elevation of another alternative embodiment of an endoscope.

FIG. 10 is an alternative embodiment of the laser in receipt of the alternative endoscope of FIG. 9.

FIG. 11 is the alternative embodiment of the endoscope in accordance with FIG. 8 without the internally received adapted.

FIG. 12 is the internally received adapter of FIG. 1.

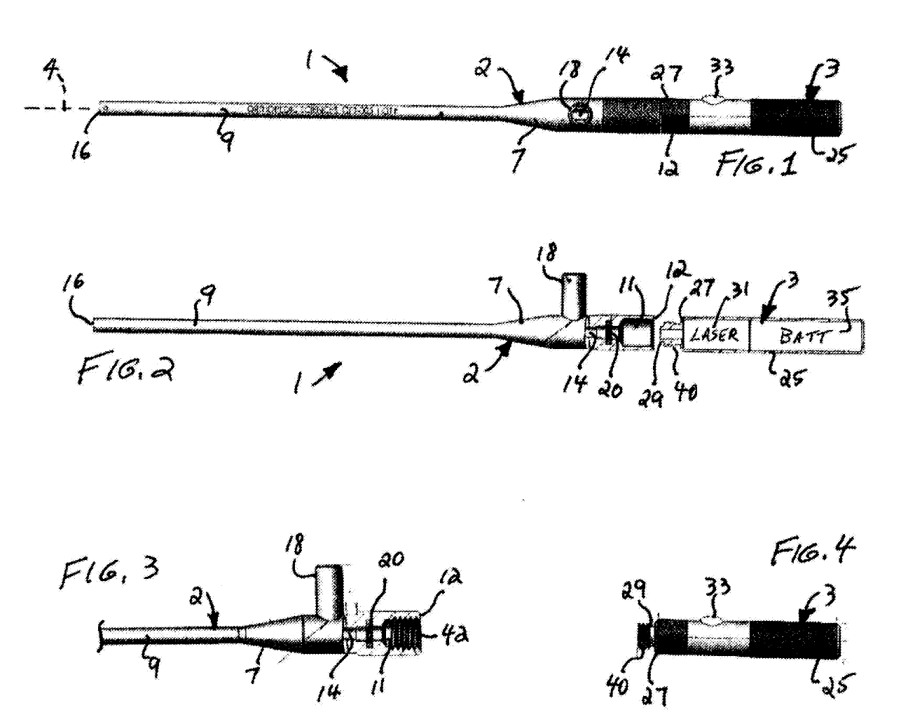

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 1 generally designates an embodiment of a laser sighting endoscopic instrument or endoscope according to the present invention. The instrument 1 generally includes an endoscopic instrument or endoscope unit 2 and a laser unit 3 removably joined with the endoscope unit 2 for sending a laser beam 4 therethrough.

The illustrated endoscopic unit 2 includes an enlarged hub or grip section 7 at a rear end from which an elongated rigid cannula 9 extends. The hub 7 has an enlarged passage 11 terminating proximally in a rear port 12. The enlarged passage 11 communicates with an elongated opening or lumen 14 and extends through the remainder of the hub 7 and the cannula 9 to a distal end 16 of the cannula 9. The hub 7 may include a side port 18 communicating with the lumen 14 or the passage 11. The hub 7 may be provided with one or more seal members or O-rings 20 to control the outflow of fluids from a surgical site through the rear port 12 when certain surgical instruments are extended through the unit 2. The endoscopic unit 2 may be any type of endoscopic instrument. The illustrated unit 2 is configured as a portal instrument which is employed to establish and maintain an open path from an incision to a surgical site. Portal instruments also provide for the insertion of endoscopic instruments toward the surgical site and are used to manage the introduction and removal of various fluids to and from the surgical site. As a portal instrument, the distal end 16 of the cannula 9 of the illustrated endoscopic unit 2 has a non-cutting circular edge.

The illustrated laser unit 3 includes a laser unit housing 25 which terminates at a distal end 27 in an attachment section, plug, or barrel 29. The housing 25 has a laser source 31 (shown diagrammatically as “LASER” in FIG. 2) such as a laser diode which generates the beam 4. The laser source 31 may include further circuitry, including a control or activation switch 33, and may be powered by a power source or battery (BATT) 35 carried in the housing 25 or by an external power supply. The control switch 33 may be a momentary push-button type of switch which causes the laser source 31 to activate as long as the switch 33 is held closed or may be a latching toggle type of switch in which a first momentary operation of the switch 33 activates the laser source 31 and a next operation of the switch deactivates the laser source 31. Alternatively, the switch 33 can be in the form of a rotary switch with a knob or the like (not shown) provided at a distal end of the housing 25. In the illustrated laser unit 3, the attachment section or plug 29 is configured as a threaded barrel having threads 40 which are configured to mate with complementary threads 42 (FIG. 4) formed in the enlarged passage 11 of the hub 7. It is foreseen that other types of junctions between the laser unit 3 and hub 7 of the endoscope unit 2 could be employed, such as a frictional fitting, a snap-in arrangement, a key and groove arrangement, a bayonet connection, a Luer fitting, or the like.

The illustrated laser unit 3 is similar in many respects to the types of laser units that are used as pointing lasers, as for use in presentations. However, the laser unit 3 is preferably smaller in overall size for convenient use with the endoscope unit 2. Such pointing lasers generate a thin beam of coherent monochromatic light and typically have a laser power output in the range of about one 1 to 5 mW (milliwatts). The laser unit 3 preferably has a laser power output at the low end of such a range to avoid any heating or other effects on tissues at the surgical site. Pointing types of lasers are available in a number of colors. For a given level of laser power, green lasers having a wavelength of about 532 nm (nanometers) appear brightest because the typical human eye is most sensitive to light in the green region of the visible spectrum. Although a 532 nm green laser source 31 is preferred in the laser unit 3, it is foreseen that other color lasers could be employed. Because even low power laser devices can cause injuries, especially to the eyes, the manufacture and approval of such devices is regulated by government agencies.

In use of the laser sighting endoscope 1, the endoscope unit 2 is inserted through an incision toward a surgical site, such as a hip joint at which a femoral head has been distracted from an acetabulum of the patient's pelvic bone. A viewing scope with a light source (not shown) may be inserted through a separate incision to provide visual triangulation of the surgical site, that is, a visual image of the site. The laser unit 3 may be attached to the endoscope unit 2, as by insertion of the attachment section 29 into the enlarged passage 11 and mating the threads 40 and 42. Typically, the visual field available to the surgeon greatly exceeds the field of reach or access using an endoscopic instrument with a rigid cannula. That is, the surgeon can see regions within the surgical site which cannot be reached for surgical operations using the rigid endoscope. In order to determine and visualize the actual field of surgical access, the surgeon activates the laser unit 3 by operation of the switch 33 to thereby radiate a laser beam through the lumen 14 of the cannula 9 into the surgical site. The surgeon can then manipulate the endoscope unit 2 to determine the available degree of freedom of the endoscope unit 2. During manipulation of the endoscope unit 2, the surgeon can visually note any potential contact with sensitive tissues, such as femoral head cartilage, by illumination of the laser beam 4 without actual contact with such tissues. Moving images of manipulation of the endoscopic instrument 1 with the laser unit 3 activated can be recorded for reference purposes. Once the surgeon has a feel for the prudent field of surgical access, the laser unit 3 can be deactivated and removed from the endoscope unit 2 and replaced with various surgical tools for carrying out surgical operations such as cutting, shaving, debriding, cauterizing, or the like.

An additional use of the laser sighting endoscope 1 includes exterior use of the endoscope in association with an external alignment system for aligning an internal component with a desired exterior reference point; the exterior reference point located a distance away from the surgical site and forming an alignment axis in parallel with an interior structure associated with the desired angular position of the interior structure in relation to the alignment axis.

It is foreseen that the sighting laser unit 3 can be replaced with a surgical laser unit (not shown) for required surgical operations. Laser units employed for surgeries tend to be much higher powered, such as in the range of about 30 to 100 watts. It is also foreseen that such a surgical laser unit could be combined with a sighting laser unit 3 with optical elements, such as a prism or prisms, employed to direct the beams therefrom through the lumen 14.

It is also foreseen that if the laser beam 4 is not aligned substantially with the axis of the lumen 14, impingement of the beam 4 with internal surfaces of the lumen 14 can cause some decollimation or dispersion of the laser beam 4. Because of the relatively short distance involved in the length of the cannula 9 and the distance from the tip 16 to tissues within the surgical site, such dispersion would not be detrimental to the function of the instrument 1. The instrument 1 could still be used to effectively determine the field of surgical access at the surgical site.

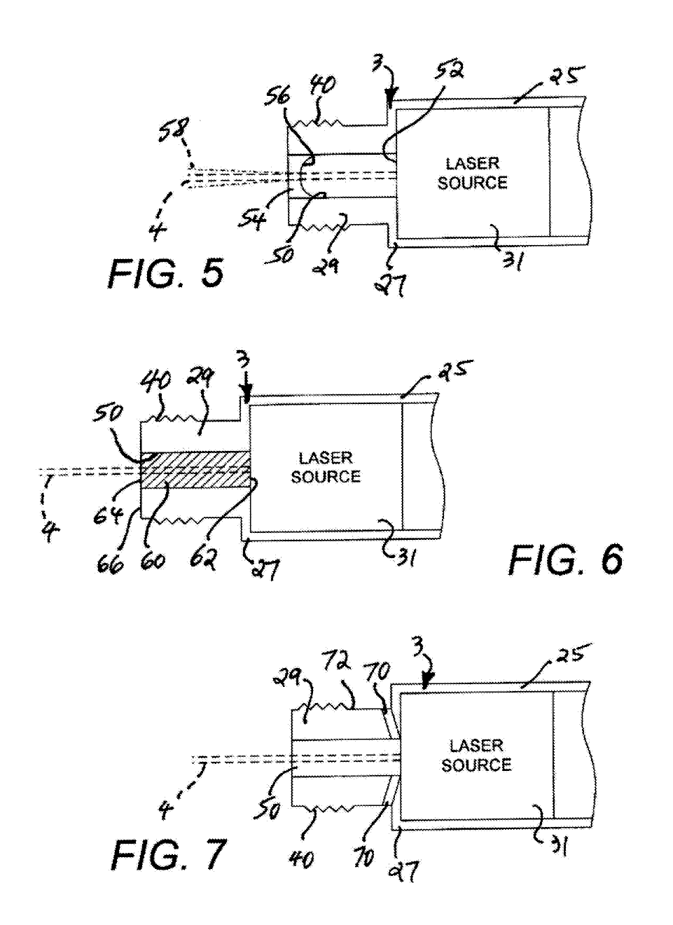

In order for the surgeon to accurately measure the field of surgical access, collimation of the laser beam 4 must be maintained from the laser source 31 to the surgical site so that the spot of illumination viewed within the surgical site is small. Thus, decollimation or divergence of the beam 4 reduces the accuracy of the measurement process. The laser beam 4 can be decollimated by refraction which occurs when the beam impinges upon curved or angled relative to the beam axis. Such a curved surface can be formed by liquids such as irrigants within the endoscope unit 2 which are typically used in endoscopic surgery. Referring to FIG. 5, a laser unit 3 is shown in which the threaded barrel 29 has a hollow bore 50. Because the bore 50 is of a small diameter and closed at a distal end 52, any liquid 54 which enters the bore 50 can only partially fill the bore 50 since there is no outlet for air initially present in the bore 50. The liquid 54 which does enter forms a meniscus 56 at the line of contact between the liquid 54 and the inner surface of the bore 50. As illustrated, the curvature of the surface of the meniscus 56 can refract portions of the beam 4, thereby causing decollimation or divergence of the beam 4. The divergence of the beam 4 is indicated in FIG. 5 at 58. It is foreseen that the meniscus 56 may cause initial convergence of the beam to a focal point (not shown) and thereafter divergence. As the diverged beam passes through the lumen 14 and exits the distal end 16 of the cannula 9, the illumination provided by the laser beam 4 may not be sufficiently focused for the surgeon to accurately judge the field of access of the endoscope 1.

FIG. 6 illustrates an embodiment of the laser unit 3 in which a cylindrical lens element 60 is positioned within the bore 50 of the barrel 29. The illustrated lens 60 has proximal and distal end surfaces 62 and 64 which are flat and which are oriented precisely perpendicular to the axis of the laser beam 4. As a consequence, impingement of the beam 4 on the surfaces 62 and 64 causes no refraction and, thus, no decollimation of the beam 4. Additionally, the distal end surface 64 is preferably flush with a distal end surface 66 of the barrel 29. Because of this, no liquid can enter the bore 50 of the barrel 29 to form a meniscus with the bore 50. The lens 60 may be formed of any optically and biologically appropriate transparent material. In use, the laser unit 3 is threaded into the rear port 12 of the endoscope unit 2, and the enlarged passage 11 and lumen 14 are filled with the irrigant, such that the irrigant fully contacts the distal end surface 64 of the lens 60. When the laser source 31 is activated, the laser beam 4 radiates through the lens 60 and the irrigant within the endoscope unit 2 to the surgical site with minimal refraction and decollimation.

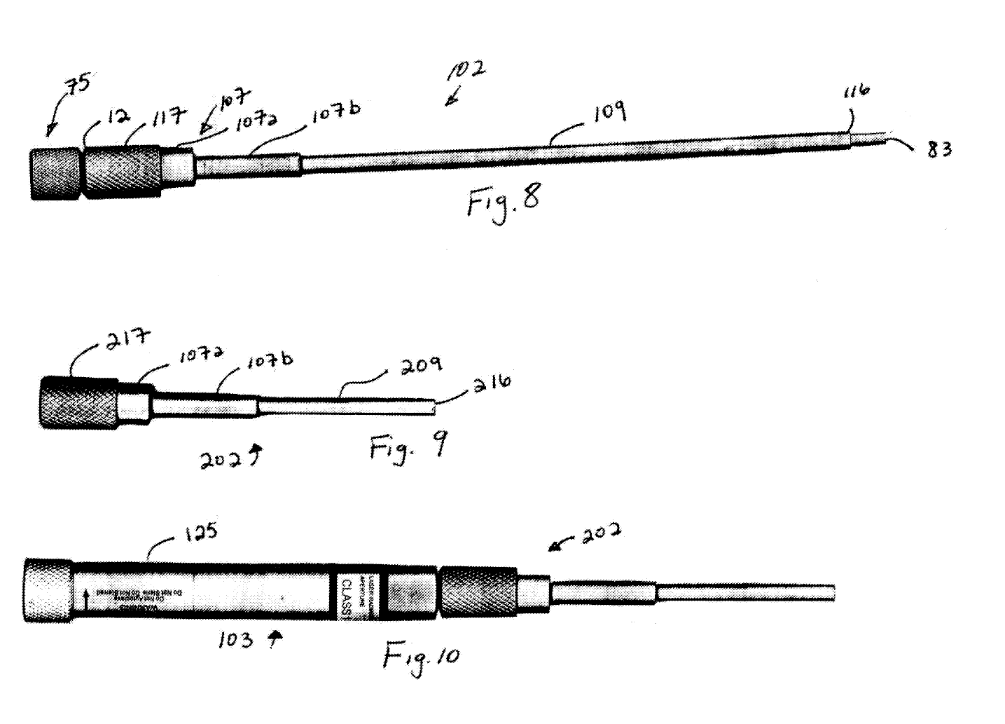

FIG. 7 illustrates an alternative embodiment of the laser unit 3 in which collimation of the laser beam 4 is maintained by the provision of air purge passages 70 are provided in the threaded barrel 29. The purge passages 70 extend radially from the bore 50 of the barrel 29 to an outer cylindrical surface 72 of the barrel. The passages 70 enable air present within the bore 50 of the barrel 29 to be pushed out by liquid entering the bore 50. In use, the barrel 29 of the laser unit 3 is threaded most of the way into the rear port 12, and the endoscope unit 2 is filled with the irrigant. The irrigant is allowed to flow into the bore 50 to completely fill it by the passage of air out the passages 70. Thereafter, the barrel 29 is fully threaded into the rear port 12 to seal the passages 70. The inner surface of the rear port 12 may be provided with a seal member (not shown) to positively close the passages 70 to prevent undesired leakage of the irrigant from the laser unit 3. When the laser source 31 is activated, the laser beam 4 passes through the irrigant within the bore 50 and thereafter through the irrigant within the endoscope unit 2. Since the bore 50 and endoscope unit 2 are completely filled with the irrigant, a consistent medium is provided for the laser beam 4, thereby avoiding decollimation of the beam 4.

FIG. 8 illustrates an alternative embodiment of an endoscopic unit 102 including an enlarged hub or grip section 107 with an outer radial surface 107a and an inner radial surface 107b located at the proximal end of a cannula 109 spaced from a distal cannula end 116. A elongated cannula support 75 with an outer radial surface 81 less than an outer radial surface associated with the cannula 109. The elongated cannula support 75 is shown in FIG. 8 extending outwardly from the distal cannula end 116 with a distal support end 83 extending outwardly therefrom. The rear port 112 of the alternative endoscopic instrument 102 is adapted for receiving the elongated cannula support with the rear port 112 including a receiving structure such as a threaded receiver (not shown) adapted for receiving a threaded end associated with a frictional grip of the elongated receiver, the frictional grip having an arcuate lip for engagement by a second arcuate lip associate with the endoscopic instrument, the arcuate lip pair presenting a v-channel therebetween. The v-channel presents a grooved surface therebetween.

As illustrated in FIG. 11, the inner radial surface 107b is adapted for receipt by an alignment instrument (not shown) associated with an external alignment system (also not shown). In one embodiment, the alignment instrument has a plurality of apertures, one of which has an inner diameter slightly greater than the inner radial surface 107b. The alignment instrument is adapted for securing the inner radial surface 107b during reciprocally movement therein, the alignment instrument aligning the elongated cannula 109 along a lateral tibial axis associated with the external alignment system and the repaired orthopedic structure. One example of an external alignment system is the DePuy P.F.C. Sigma. PR-F system available from DePuy International Ltd. Sigma and P.F.C are trademarks of DePuy Orthopedics, Inc.

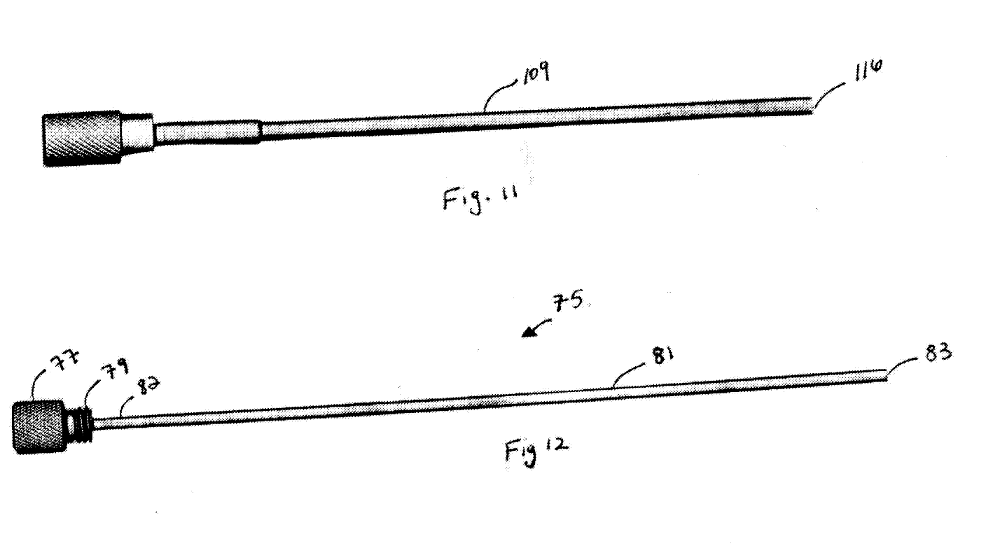

A second alternative elongated endoscope 202 is illustrated in FIGS. 9-10 with a shortened elongated cannula 209 extending towards a distal end 216 from a proximal endoscopic end 217. The laser unit 103 is illustrated in FIG. 10 with a housing 125 containing the laser source 31, the laser beam extending from the laser source 31 through the second alternative endoscopic instrument 202. As illustrated, the second alternative endoscopic instrument 202 is mechanically aligned with the housing 125 surrounding the laser unit 103 for transmission of the laser beam therethrough. In general, the radial surface extending outwardly from the elongated rigid cannula 9, 109 and 209 is rigid and is visually aligned between the rear port 12 and respective distal ends 16, 116, 216 for transmission of the laser beam from the laser source 31 therethrough. Depending on the operating conditions, the shortened elongated cannula 209 may be utilized, for example, if the elongated cannula is damaged or is the distance the laser source 31 and the desired point of alignment is relatively near.

As further illustrated in FIG. 11, the hub 107 has an enlarged passage 111 terminating proximally in a rear port 112. The enlarged passage 111 communicates with an elongated opening or lumen 114 and extends through the remainder of the hub 107 and the cannula 109 to a distal end 116 of the cannula 109. The hub 107 may have a threaded end (not shown) for receipt of the elongated cannula support 75 of FIG. 12. The elongated cannula support 75 presents an outer radial surface 81 adapted for receipt by inner radial surface of the elongated cannula 109 which is supported therealong. In this way, the elongated cannula 109 resists deflection during operation of the endoscopic instrument 102 for visual alignment of the laser beam traveling therethrough.

The elongated cannula support 75 includes a frictional grip associated with a proximal support end 77 with a threaded structure 79 cylindrically extending towards a proximal end associated with the outer radial surface 81. The elongated cannula support 75 is adapted for cylindrical support of the elongated cannula 109 for alignment and transmission of the laser beam therethrough.

It is to be understood that while certain forms of the present invention have been illustrated and described herein, it is not to be limited to the specific forms or arrangement of parts described and shown.

What is claimed and desired to be secured by Letters Patent is:

1. A laser sighting endoscopic instrument for positioning a laser beam at reference point in association with a surgical site, the instrument comprising:
an endoscopic portal having a laser unit adapted for attachment to the endoscopic portal in alignment with an axis of a lumen extending along an elongated cannula
a proximal hub extending rearwardly from said endoscopic portal, the lumen extending therefrom to a distal tip associated with said lumen and spaced from said proximal hub, the hub being adapted for removable receipt of said laser unit,
said laser unit and said hub in cooperation with each other for position of a laser beam exiting said laser unit along the longitudinal axis of said cannula.

2. The laser sighting endoscopic instrument of claim 1 wherein said laser unit further includes an attachment section having a threaded barrel for secure alignment of said laser beam through said lumen to a said reference point.

3. The laser sighting endoscopic instrument of claim 1 the laser beam is transmitted to a reference point a distance from the surgical site thereby presenting a visual alignment axis.

4. The laser sighting endoscopic instrument of claim 1 wherein the laser includes a lens element positioned with a bore of a threaded barrel associated with a proximal end.

5. The laser sighting endoscopic instrument of claim 1 further the wherein the lumen has an elongated cannula presenting an outer radial surface adapted for receipt by an external alignment system.

6. The laser sighting endoscopic instrument of claim 5 wherein the elongated cannula has an inner radial surface adapted for receipt of an elongated cannula support therethrough.
